(19) United States
(12) Patent Application Publication
(10) Pub. No.: US 2026/0200777 A1
(43) Pub. Date: Jul. 16, 2026

(54) METHOD FOR TREATING REFRACTORY WASTEWATER PRODUCED IN STEPS FOR MANUFACTURING PET RAW MATERIAL

(71) Applicant: NATIONAL INSTITUTE OF ADVANCED INDUSTRIAL SCIENCE AND TECHNOLOGY, Tokyo (JP)

(72) Inventors: Kyohei KURODA, Ibaraki (JP); Takashi NARIHIRO, Ibaraki (JP); Masaru Konishi NOBU, Ibaraki (JP); Masayoshi YAMADA, Kagoshima (JP); Masahito YAMAUCHI, Kagoshima (JP)

(21) Appl. No.: 19/138,374

(22) PCT Filed: Dec. 15, 2023
(86) PCT No.: PCT/JP2023/045156

§ 371 Date: Jun. 12, 2025

(30) Foreign Application Priority Data
Dec. 16, 2022 (JP) 2022-201276

(51) Int. Cl. C02F 3/28; C02F 101/30; C02F 103/36

(52) U.S. Cl. CPC C02F 3/2846; C02F 2101/30; C02F 2103/365

(57) ABSTRACT

Provided is a method of collective treatment of organic wastewater discharged in processes of manufacturing purified terephthalic acid (PTA) and dimethyl terephthalate (DMT) which are raw materials for polyethylene terephthalate (PET). The inventors found that collective anaerobic biological wastewater treatment of PTA manufacturing wastewater and DMT manufacturing wastewater enables both the wastewaters to be collectively treated with efficiency more than the conventional efficiency of treatment of PTA manufacturing wastewater. The present invention was thus accomplished.

### FIELD

The present invention relates to a method of collective treatment of high-concentration organic wastewater discharged in processes of manufacturing purified terephthalic acid (PTA) and dimethyl terephthalate (DMT) which are raw materials for polyethylene terephthalate (PET).

### BACKGROUND

Polyethylene terephthalate (PET) has been produced worldwide, principally as fibers and materials for beverage PET bottles. About 33 Mton of PET was produced in 2015, accounting for 10% of total plastic production with an average annual growth rate of 11% (NPLS 1 to 3). PET has been principally utilized in PET bottle beverage containers, and 488 billion PET bottles were produced in the world in 2016 (NPL 4).

PET is manufactured by polymerization of purified terephthalic acid (PTA) or dimethyl terephthalate (DMT) with ethylene glycol. PTA or DMT is manufactured from petroleum-derived p-xylene, and a large amount of wastewater with high organic loading (3 to 10 tons per ton of manufactured PTA/DMT) is discharged in a manufacturing process thereof (NPLs 5 to 7). Typical PTA manufacturing wastewater (5 to 20 g COD·L⁻¹) contains high concentrations (0.6 to 6 mM) of aromatic compounds (terephthalic acid (TA), benzoic acid (BZ), isophthalic acid (IA), orthophthalic acid (OA), and p-toluic acid (PT)), more than 20 mM methanol (MT), and acetic acid (AC) (NPLs 5 and 8). DMT manufacturing wastewater (24 to 162 g COD·L-1) contains AC and MT (accounting for 60% or more of COD) as main components, and also contains other components such as formic acid (FM) and formaldehyde (NPLs 7 and 9).

PET is a material in extremely high demand, and therefore, the market size thereof is expanding worldwide. As a result, the discharge amounts of PTA and DMT manufacturing wastewaters in need of treatment are also expected to be further increased. These wastewaters are often treated using anaerobic biological wastewater treatment methods.

Anaerobic wastewater treatment technologies such as upflow anaerobic sludge blanket (UASB) and expanded granular sludge bed (EGSB) processes have been applied in treatment of PTA manufacturing wastewater (NPL 10). To date, various treatment systems have been studied to optimize PTA wastewater treatment technologies (for example, mesophilic (37° C.) two-stage UASB systems: organic loading rate (OLR) of 25 kg·m⁻³ day “1, COD removal efficiency of 80%, synthetic PTA wastewater (TA, BZ, and AC); temperature-staged UASB systems (1st: 55° C. and 2nd: 37° C.). OLR of 4.45 kg·m⁻³ day⁻¹, COD removal efficiency of 96%, and synthetic PTA wastewater (TA, PT, BZ, OA, IA, and AC)) (NPLs 5 and 11). In the full-scale manufacturing process, DMT wastewater (6.5 to 33.8 gCOD·L⁻¹) was anaerobically treated by UASB- and EGSB-type reactors at 7 to 13.2 kg·m⁻³ day⁻¹ OLR (COD removal efficiency of 70 to 95%) (NPL 12), whereas only one study relevant to aerobic treatment of DMT wastewater (110 to 162 gCOD·L⁻¹) used a lab-scale column reactor with porous plastic biomass support particles (14 kg·m⁻³ day⁻¹ OLR, COD removal efficiency of 90%) (NPL 9).

Microbial degradation of an aromatic compound contained in PTA manufacturing wastewater is not easy. Therefore, designs with prolonged treatment time periods and the sufficient footprints of wastewater treatment systems have been implemented to satisfy effluent standards after wastewater treatment, whereby excessive costs for introduction and operation have been added. Accordingly, technological development for promoting the efficiency of treatment of PTA manufacturing wastewater can respond to requests from society with the increasing amount of consumed PET, and is expected to be highly demanded.

### CITATION LIST

### Non Patent Literature

[NPL 1]: Barnard, E., Rubio Arias, J. J., Thielemans, W., 2021. Chemolytic depolymerisation of PET: A review. Green Chem. 23, 3765-3789
[NPL 2]: Geyer, R., Jambeck, J. R., Law, K. L., 2017. Production, use, and fate of all plastics ever made. Sci. Adv. 3, 25-29
[NPL 3]: Stubbins, A., Law, K. L., Munoz, S. E., Bianchi, T. S., Zhu, L., 2021. Plastics in the Earth system. Science 373, 51-55
[NPL 4]: Volanti, M., Cespi, D., Passarini, F., Neri, E., Cavani, F., Mizsey, P., Fozer, D., 2019. Terephthalic acid from renewable sources: Early-stage sustainability analysis of a bio-PET precursor. Green Chem. 21, 885-896
[NPL 5]: Kleerebezem, R., Beckers, J., Hulshoff Pol, L. W., Lettinga, G., 2005. High rate treatment of terephthalic acid production wastewater in a two-stage anaerobic bioreactor. Biotechnol. Bioeng. 91, 169-179
[NPL 6]: Ma, K. li, Li, X. kun, Wang, K., Ren, Y. hui, Chu, Z. rui, Zhang, J., 2017. Role of temperature on microbial community profiles in an anaerobic bioreactor for treating PTA wastewater. Chem. Eng. J. 308, 256-263
[NPL 7]: Yasin, O., Zelal, I., Nadir, D., 2020. Acetic acid and methanol recovery from dimethyl terephthalate process wastewater using pressure membrane and membrane distillation processes. J. Water Process Eng. 38
[NPL 8]: Kuroda, K., Nobu, M. K., Mei, R., Narihiro, T., Bocher, B. T. W., Yamaguchi, T., Liu, W.-T., 2016. A single-granule-level approach reveals ecological heterogeneity in an upflow anaerobic sludge blanket reactor. PLOS One 11
[NPL 9]: Shah, S. S., Desai, J. D., Ramakrishna, C., Bhatt, N. M., 1998. Aerobic biotreatment of wastewater from dimethyl terephthalate plant using biomass support particles. J. Ferment. Bioeng. 86, 215-219
[NPL 10]: Kleerebezem, R., Macarie, H., 2003. Treating industrial wastewater: Anaerobic digestion comes of age. Checmical Eng. 110, 56-64
[NPL 11]: Ma, K. li, Li, X. kun, Bao, L. lin, 2019. Influence of organic loading rate on purified terephthalic acid wastewater treatment in a temperature staged anaerobic treatment (TSAT) system: Performance and metagenomic characteristics. Chemosphere 220, 1091-1099
[NPL 12]: Macarie, H., 2000. Overview of the application of anaerobic treatment to chemical and petrochemical wastewaters. Water Sci. Technol. 42, 201-214
[NPL 13]: Kuroda, K., Chosei, T., Nakahara, N., Hatamoto, M., Wakabayashi, T., Kawai, T., Araki, N., Syutsubo, K., Yamaguchi, T., 2015. High organic loading treatment for industrial molasses wastewater and microbial community shifts corresponding to system development. Bioresour. Technol. 196, 225-234
[NPL 14]: Kleerebezem, R., Pol, L. W. H., Lettinga, G., 1999. The Role of Benzoate in Anaerobic Degradation of Terephthalate. Appl. Environ. Microbiol. 65, 1161-1167
[NPL 15]: Ma, K., Zhang, X., Shang, Y., Zhu, Z., Li, Xilin, Li, Xiaoling, Li, Xiangkun, 2020. Improved purified terephthalic acid wastewater treatment using combined UAFB-SBR system: At mesophilic and ambient temperature. Chemosphere 247, 125752
[NPL 16]: Liang, D. W., Zhang, T., Fang, H. H. P., He, J., 2008. Phthalates biodegradation in the environment. Appl. Microbiol. Biotechnol. 80, 183-198
[NPL 17]: Joung, J. Y., Lee, H. W., Choi, H., Lee, M. W., Park, J. M., 2009. Influences of organic loading disturbances on the performance of anaerobic filter process to treat purified terephthalic acid wastewater. Bioresour. Technol. 100, 2457-2461
[NPL 18]: Ma, X. chen, Li, X. kun, Wang, X. wei, Liu, G. ge, Zuo, J. long, Wang, S. tao, Wang, K., 2020. Impact of salinity on anaerobic microbial community structure in high organic loading purified terephthalic acid wastewater treatment system. J. Hazard. Mater. 383, 121132
[NPL 19]: Garg, K. K., Prasad, B., 2017. Treatment of toxic pollutants of purified terephthalic acid waste water: A review. Environ. Technol. Innov. 8, 191-217

### SUMMARY

### Technical Problem

The present invention relates to a method of collective treatment of high-concentration organic wastewater discharged in processes of manufacturing purified terephthalic acid (PTA) and dimethyl terephthalate (DMT) which are raw materials for polyethylene terephthalate (PET).

### Solution to Problem

In recent years, not only petroleum-derived raw materials but also new PET manufacturing process technologies such as conversion of PET waste into raw materials (monomerization) and more environmentally friendly bio-based PET manufacturing (biomass-derived p-xylene manufacturing) have become popular, and the construction of plants in new business forms has been socially expected (NPLs 1 and 4). Currently, PTA and DMT have been manufactured in separate plants, and therefore, processes of treatment of wastewater generated in the manufacturing processes thereof have also been separately placed and operated. However, a demand for developing a technology of simultaneously treating wastewaters with different properties, discharged from different processes, in a single process is expected to increase in the future. From the viewpoint of effective wastewater treatment (for example, compact area and low energy requirements), cotreatment of PTA and DMT wastewaters is considered to be advantageous.

The inventors found that collective anaerobic biological wastewater treatment of PTA manufacturing wastewater and DMT manufacturing wastewater enables both the wastewaters to be collectively treated with efficiency more than the conventional efficiency of treatment of PTA manufacturing wastewater. In an unexpected improvement in such treatment efficiency, methanol and formic acid components in the DMT manufacturing wastewater are presumed to contribute to the acceleration of the degradation of organic compounds in the PTA manufacturing wastewater.

In the present invention, a long-term continuous treatment experiment was conducted for 518 days by increasing the concentration of organic substance in a stepwise manner in an upflow anaerobic sludge blanket (UASB) reactor produced on a laboratory scale, and the concentration of organic substance in mixed wastewater according to the present invention, and treatment conditions under which 90% or more thereof can be stably degraded were specified. In such specific conditions, the rate of the degradation of the organic substance was shown to be 1.2 to 4.4 times those in conventional PTA manufacturing wastewater treatment reactors of the conventionally was shown. Since the amounts of existing methanogenic archaea utilizing formic acid and hydrogen and existing anaerobic symbiotic bacteria degrading an aromatic compound were increased by adding formic acid into the reactor, it was presumed that the addition of the components contained in the DMT manufacturing wastewater to the PTA manufacturing wastewater resulted in the activation of the anaerobic symbiotic bacteria and methanogenic archaea essential for the degradation of the aromatic compound due to the interaction thereof in the UASB reactor, and high treatment efficiency was shown. As described above, efficient collective treatment of the composite wastewater of the PTA manufacturing wastewater and the DMT manufacturing wastewater was achieved.

Accordingly, the present application provides the following inventions.
1. A method of treating organic wastewater containing an aromatic compound, the method comprising:
(a) a step of performing addition of formic acid and methanol to the organic wastewater to make mixed wastewater; and
(b) a step of subjecting the mixed wastewater to anaerobic biological wastewater treatment to degrade an organic substance in the mixed wastewater.
2. The method according to item 1, wherein the organic wastewater is purified terephthalic acid (PTA) manufacturing wastewater.
3. The method according to item 1, wherein the addition of the formic acid and the methanol in the step (a) is performed by addition of dimethyl terephthalate (DMT) manufacturing wastewater.
4. The method according to item 1, wherein the aromatic compound contained in the organic wastewater is one or two or more compounds selected from a group consisting of orthophthalic acid, benzoic acid, terephthalic acid, isophthalic acid, p-toluic acid, and salts and esters thereof.
5. A method of treating purified terephthalic acid (PTA) manufacturing wastewater, the method comprising:
(a) a step of adding dimethyl terephthalate (DMT) manufacturing wastewater to the PTA manufacturing wastewater to make mixed wastewater; and
(b) a step of subjecting the mixed wastewater to anaerobic biological wastewater treatment to degrade an organic substance in the mixed wastewater, wherein the DMT manufacturing wastewater contains formic acid and methanol.
6. The method according to item 5, wherein an aromatic compound contained in the PTA manufacturing wastewater is one or two or more compounds selected from a group consisting of orthophthalic acid, benzoic acid, terephthalic acid, isophthalic acid, p-toluic acid, and salts and esters thereof.
7. The method according to any one of items 1 to 6, wherein a concentration of the formic acid in the mixed wastewater prepared in the step (a) is 1 to 3% with respect to a total COD concentration.
8. The method according to any one of items 1 to 6, wherein a concentration of the methanol in the mixed wastewater prepared in the step (a) is 7 to 13% with respect to a total COD concentration.
9. The method according to any one of items 1 to 6, wherein the method further comprises (c) a step of separating, from the mixed wastewater, methane generated by degrading the organic substance in the step (b).
10. The method according to any one of items 1 to 6, wherein the anaerobic biological wastewater treatment in the step (b) is performed using a reactor filled with methanogenic sludge.
11. The method according to item 10, wherein wastewater is treated in upflow in the reactor filled with the methanogenic sludge.
12. The method according to item 10, wherein the methanogenic sludge comprises methanogenic archaea and anaerobic symbiotic bacteria.
13. The method according to item 12, wherein the methanogenic archaea comprise one or two or more kinds of archaea selected from a group consisting of archaea belonging to genera Methanobacterium, Methanolinea, Methanoregula, Methanothrix, Methanomethylovorans, and Methanomassiliicoccus.
14. The method according to item 12, wherein the anaerobic symbiotic bacteria comprise one or two or more kinds of bacteria selected from a group consisting of bacteria belonging to genera Syntrophus, Syntrophorhabdus, and Pelotomaculum.

### BRIEF DESCRIPTION OF DRAWINGS

FIG. 1 illustrates a schematic view of an upflow anaerobic sludge blanket (UASB) used in this study.

FIG. 2 illustrates total COD concentration and removal efficiency (A), soluble COD concentration and removal efficiency (B), organic loading rate (OLR) (C), the amount of produced gas and the cumulative amount of produced gas (D), suspended solid (SS) concentration and removal efficiency (E), volatile SS (VSS) concentration and removal efficiency (F), VSS/SS ratio (G), and the time course of pH in an upflow anaerobic sludge blanket (UASB) used in this study (H).

FIG. 3 illustrates a methane conversion efficiency based on the correlation between a methane production rate and an organic substance removal rates in an upflow anaerobic sludge blanket (UASB) used in this study.

FIG. 4 illustrates COD mass balances in an upflow anaerobic sludge blanket (UASB) reactor during phases 1 to 8. COD concentrations were calculated using the average value of three operational days in each phase as follows: Days 75, 78, and 81 for phase 1; Days 138, 141, and 145 for phase 2; Days 169, 173, and 176 for phase 3; Days 243, 247, and 258 for phase 4; Days 299, 320, and 323 for phase 5; Days 366, 370, and 384 for phase 6; Days 414, 422, and 429 for phase 7; and Days 509, 516, and 518 for phase 8. Inf: influent; Eff: effluent; OA: orthophthalic acid; TA: terephthalic acid; IA: isophthalic acid; BZ: benzoic acid; PT: p-toluic acid; FM: formic acid; AC: acetic acid; PP: propionic acid; and unknown: remaining unclassified total COD concentration.

### DESCRIPTION OF EMBODIMENTS

The present invention is described in detail below with reference to specific embodiments. However, the present invention is not bound by the following embodiments, but can be carried out in an optional form without departing from the gist of the present invention.

One aspect of the present invention relates to a method of treating organic wastewater containing an aromatic compound, the method comprising: (a) a step of performing addition of formic acid and methanol to the organic wastewater to make mixed wastewater; and (b) a step of subjecting the mixed wastewater to anaerobic biological wastewater treatment to degrade an organic substance in the mixed wastewater.

In the method of treating organic wastewater of the present invention, the organic wastewater to be treated is not particularly limited, and the organic wastewater containing an aromatic compound is acceptable. The kind of the aromatic compound contained in the organic wastewater is not limited, and the aromatic compound preferably contains one or two or more compounds selected from the group consisting of orthophthalic acid, benzoic acid, terephthalic acid, isophthalic acid, p-toluic acid, and salts and esters thereof.

In the method of treating organic wastewater of the present invention, examples of the organic wastewater to be treated include purified terephthalic acid (PTA) manufacturing wastewater or dimethyl terephthalate (DMT) manufacturing wastewater. Each of PTA and DMT is a synthetic raw material for polyethylene terephthalate (PET), and a large amount of PET is principally produced as a material for a beverage PET bottle. Therefore, large amounts of PTA and DMT which are raw materials therefor are also produced, and thus, large amounts of PTA and DMT manufacturing wastewaters are also discharged. Herein, “purified” terephthalic acid means terephthalic acid having a purity of 99% by mass or more, unless otherwise specified.

The PTA manufacturing wastewater commonly contains, without limitation, an aromatic compound at a high concentration, methanol (MT), and acetic acid (AC). Examples of the aromatic compound include aromatic carboxylic acids such as terephthalic acid (TA), benzoic acid (BZ), isophthalic acid (IA), orthophthalic acid (OA), and p-toluic acid (PT), or salts or esters thereof. Examples of the salts of such aromatic compounds include alkali metal salts of sodium, potassium, and the like, and alkali earth metal salts of calcium, magnesium, and the like. The esters of such aromatic compounds include alkyl esters such as methyl esters and ethyl esters.

The aromatic compound contained in the PTA manufacturing wastewater is not easily degraded by anaerobic biological wastewater treatment commonly used for treating such wastewater, and a cost and time are required to achieve desired degradation. In the present invention, mixed wastewater obtained by adding formic acid and methanol is subjected to the anaerobic biological wastewater treatment in order to more efficiently treat the organic wastewater containing such an aromatic compound.

The present invention is based on the surprising findings in that the degradation of the aromatic compound by the anaerobic biological wastewater treatment is accelerated by adding formic acid and methanol. Such findings show the effectiveness of collective treatment of PTA manufacturing wastewater containing an aromatic compound and DMT manufacturing wastewater containing formic acid and methanol, and can also be applied to treatment of other wastewater containing these organic compounds. Alternatively, an aspect in which predetermined amounts of formic acid and methanol are added in degradation treatment of wastewater containing an aromatic compound can also be intended.

In a preferred aspect, the organic wastewater containing the aromatic compound is PTA manufacturing wastewater, and the addition of the formic acid and the methanol is performed by addition of DMT manufacturing wastewater. In other words, an aspect of the present invention relates to a method of treating purified terephthalic acid (PTA) manufacturing wastewater, the method comprising: (a) a step of adding dimethyl terephthalate (DMT) manufacturing wastewater to the PTA manufacturing wastewater to make mixed wastewater; and (b) a step of subjecting the mixed wastewater to anaerobic biological wastewater treatment to degrade an organic substance in the mixed wastewater.

The adequate amounts of added formic acid and methanol to the organic wastewater containing the aromatic compound can vary depending on the conditions of the anaerobic biological wastewater treatment by which the mixed wastewater is degraded, for example, the configuration and operating conditions of a UASB reactor, the microbial activity profile of filled methanogenic sludge, and the like. However, those skilled in the art can determine the adequate amounts of added formic acid and methanol by using an adequate preliminary study without requiring excessive trial and error.

Specifically, the amount of added formic acid with respect to the organic wastewater is preferably adjusted so that the concentration (mg/L) of formic acid in the obtained mixed wastewater is typically 1% or more, especially 1.2% or more, and typically 3% or less, especially 2.8% or less, with respect to a total COD concentration (mg/L), without limitation.

The amount of added methanol with respect to the organic wastewater is preferably adjusted so that the concentration (mg/L) of methanol in the obtained mixed wastewater is typically 7% or more, especially 7.5% or more, and typically 13% or less, especially 12% or less, with respect to the total COD concentration (mg/L), without limitation.

The anaerobic biological wastewater treatment is a common technology to treat organic wastewater such as PTA manufacturing wastewater or DMT manufacturing wastewater in the present invention. The organic wastewater to be treated and anaerobic microorganisms are kept under an anaerobic condition in the reactor, and an organic substance in the wastewater is degraded into a harmless or useful substance such as methane or carbon dioxide by the anaerobic microorganisms. A condition under which the anaerobic biological wastewater treatment is performed can be set as appropriate by those skilled in the art depending on the kind and content of the organic compound to be degraded, such as the aromatic compound contained in the organic wastewater subjected to the treatment, and treatment efficiency to be achieved.

A reactor filled with methanogenic sludge can be used as means for the anaerobic biological wastewater treatment, without limitation. Especially, the wastewater is preferably treated in upflow in the reactor filled with the methanogenic sludge. The kind of such a reactor is not limited. However, an upflow anaerobic sludge blanket (UASB) reactor can be used in accordance with an aspect. A microbial aggregate (methanogenic sludge) at a high concentration is kept in the UASB reactor, and the wastewater is treated in upflow from the lower portion thereof, whereby the organic substance can be degraded into methane and carbon dioxide to collect a methane gas as energy. In another aspect, means obtained by coupling a UASB reactor and an upflow anaerobic fluidized bed (UAFB) reactor, an expanded granular sludge bed (EGSB) reactor, an internal circulation (IC) reactor, or the like can be used as means for the anaerobic biological wastewater treatment of the present invention.

In the present invention, the methanogenic sludge means a sludgy solid that supports a microorganism group that degrades an organic compound to generate methane. In the anaerobic biological wastewater treatment of the present invention, the microorganism group existing in the methanogenic sludge filled into the reactor degrades the organic compound targeted for treatment of the aromatic compound and the like in the organic wastewater to generate methane. The microorganism group in the methanogenic sludge preferably principally includes anaerobic symbiotic bacteria and methanogenic archaea, without limitation.

The anaerobic symbiotic bacteria mean microorganisms that form a microorganism symbiosis system together with methanogenic archaea and degrade an organic acid such as an aromatic compound such as terephthalic acid or benzoic acid, propionic acid, or butyric acid under an oxygen-free environment. Examples of the anaerobic symbiotic bacteria that are contained in the methanogenic sludge and contribute to the degradation of the aromatic compound in the present invention include, but are not limited to, bacteria belonging to the genera Syntrophus, Syntrophorhabdus, and Pelotomaculum.

The methanogenic archaea mean archaea that produce a methane gas from hydrogen, carbon dioxide, methanol, formic acid, acetic acid, or the like under an oxygen-free environment. Examples of the methanogenic archaea that are contained in the methanogenic sludge and has a symbiotic relationship with the anaerobic symbiotic bacteria in the present invention include, but are not limited to, archaea belonging to the genera Methanobacterium, Methanolinea, Methanoregula, Methanothrix, Methanomethylovorans, and Methanomassiliicoccus.

The degradation of the aromatic compound by the anaerobic symbiotic bacteria does not proceed in an environment in which the concentration of hydrogen is high. However, the coexistence of hydrogen-utilizing microorganisms such as methanogenic archaea allows hydrogen to be immediately removed and an environment with a low concentration of hydrogen to be maintained, whereby the degradation of the aromatic compound is operated. Accordingly, the symbiotic relationship between the anaerobic symbiotic bacteria and the methanogenic archaea is important for the degradation of the aromatic compound and the production of a methane gas in the anaerobic biological wastewater treatment of the present invention.

In the method of treating organic wastewater of the present invention, the efficiency of the degradation of the aromatic compound by the anaerobic biological wastewater treatment is improved in the presence of formic acid and methanol. This is presumed to be caused by the dissimilation of added formic acid and methanol to activate the methanogenic archaea, whereby an environment with a low concentration of hydrogen, accelerating the degradation of the aromatic compound by the anaerobic symbiotic bacteria, is maintained.

In accordance with one aspect of the present invention, the method of treating organic wastewater may further comprise (c) a step of separating, from the mixed wastewater, methane generated by degrading the organic substance in the step (b). Means of separating the methane from the mixed wastewater is not particularly limited, and optional means well known to those skilled in the art may be used. The separated methane is collected, and can be utilized as biomass energy such as a heat source, a power source, or electricity generation.

### EXAMPLES

The present invention is described in more detail below with reference to Examples. However, these examples are only examples conveniently described strictly for descriptive purposes, and the present invention is not limited to these examples in any sense.

### Preparation of Mixed Wastewater of PTA and DMT Manufacturing Wastewaters

PTA/DMT manufacturing wastewater was prepared according to Table 1. Operation stages were denoted as 1 to 8 according to the component and concentration of a substrate. The substrate was dissolved in city water heated by using a hot-plate-type magnetic stirrer (ASHS-1HP manufactured by AS ONE Corporation). Inorganic compounds (gL⁻¹: Na₂SO₄, 0.24; MgSO₄, 0.21; CaCl₂·2H₂O, 0.15; KCl, 0.30; KH₂PO₄, 0.15; NH₄Cl, 0.11) and minerals (mg·L⁻¹: FeSO₄·7H₂O, 7.0; CoCl₂·6H₂O, 0.17; ZnSO₄·7H₂O, 0.15; H₃BO₃, 0.060; MnCl₂·4H₂O, 0.42; NiCl₂·6H₂O, 0.040; CuCl₂·2H₂O, 0.027; Na₂MoO₄·2H₂O, 0.025) were added into the PTA/DMT manufacturing wastewater. The pH of the PTA/DMT manufacturing wastewater was adjusted to about 7.0 with 2M HCl and 2M NaOH.

TABLE 1
Summary of Influent Wastewater Components Used in This Study

| | Phase | | | | | | | |
|---|---|---|---|---|---|---|---|---|
| | 1 | 2 | 3 | 4 | 5 | 6 | 7 | 8 |
| | Days | | | | | | | |
| | 1-81 | 82-146 | 147-183 | 184-268 | 269-323 | 324-386 | 387-438 | 439-518 |
| | COD concentration [mgCOD/L] | | | | | | | |
| | 2,023 | 3,036 | 3,536 | 3,533 | 4,605 | 5,605 | 7,005 | 8,500 |
| Acetic acid (mgCOD/L) | 450 | 675 | 675 | 675 | 925 | 1,125 | 1,325 | 1,540 |
| Benzoic acid (mgCOD/L) | 481 | 722 | 722 | 780 | 1,020 | 1,220 | 1,420 | 1,635 |
| Orthophthalic acid (mgCOD/L) | 96.4 | 145 | 145 | 157 | 300 | 420 | 620 | 835 |
| Terephthalic acid (mgCOD/L) | 361 | 542 | 742 | 801 | 95 | 1,070 | 1,270 | 1,485 |
| Isophthalic acid (mgCOD/L) | 48.2 | 72 | 72 | 78 | 220 | 340 | 540 | 755 |
| P-toluic acid (mgCOD/L) | 346 | 520 | 520 | 562 | 710 | 830 | 1,030 | 1,245 |
| Methanol (mgCOD/L) | 240 | 360 | 360 | 360 | 360 | 480 | 680 | 895 |
| Dimethyl terephthalate (mgCOD/L) | —¹ | — | 300 | 20 | 20 | 20 | 20 | 20 |
| Formic acid (mgCOD/L) | — | — | — | 100 | 100 | 100 | 100 | 100 |

¹No substrate.

TABLE 2
Summary of Composition of Influent Wastewater Used in This Study (Percentage)

| | Phase | | | | | | | |
|---|---|---|---|---|---|---|---|---|
| | 1 | 2 | 3 | 4 | 5 | 6 | 7 | 8 |
| | Days | | | | | | | |
| | 1-81 | 82-146 | 147-183 | 184-268 | 269-323 | 324-386 | 387-438 | 439-518 |
| | COD concentration [mgCOD/L] | | | | | | | |
| | 2,023 | 3,036 | 3,536 | 3,533 | 4,605 | 5,605 | 7,005 | 8,500 |
| Acetic acid (%) | 22.2 | 22.2 | 19.1 | 19.1 | 20.1 | 20.1 | 18.9 | 18.1 |
| Benzoic acid (%) | 23.8 | 23.8 | 20.4 | 22.1 | 22.1 | 21.8 | 20.3 | 19.2 |
| Orthophthalic acid (%) | 4.8 | 4.8 | 4.1 | 4.4 | 6.5 | 7.5 | 8.9 | 9.8 |
| Terephthalic acid (%) | 17.8 | 17.9 | 21.0 | 22.7 | 20.6 | 19.1 | 18.1 | 17.5 |
| Isophthalic acid (%) | 2.4 | 2.4 | 2.0 | 2.2 | 4.8 | 6.1 | 7.7 | 8.9 |
| P-toluic acid (%) | 17.1 | 17.1 | 14.7 | 15.9 | 15.4 | 14.8 | 14.7 | 14.6 |
| Methanol (%) | 11.9 | 11.9 | 10.2 | 10.2 | 7.8 | 8.6 | 9.7 | 10.5 |
| Dimethyl terephthalate (%) | —¹ | — | 8.5 | 0.6 | 0.4 | 0.4 | 0.3 | 0.2 |
| Formic acid (%) | — | — | — | 2.8 | 2.2 | 1.8 | 1.4 | 1.2 |

¹No substrate.

### Conditions for Operating Reactor

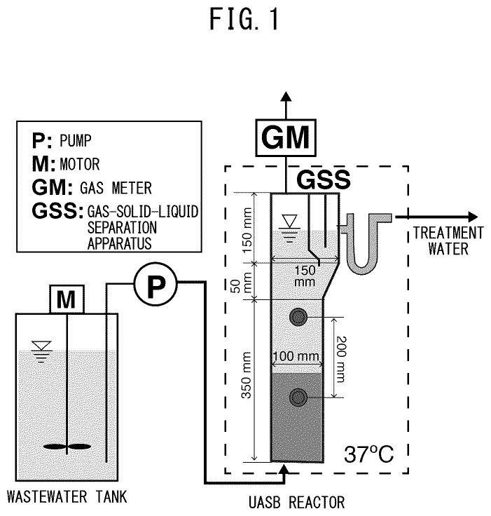

A schematic view of the UASB reactor used in Examples is illustrated in FIG. 1. The UASB reactor was installed in an incubator (MIR-554-PJ, manufactured by PHC), maintained at 37° C. The width, depth, and height of the UASB reactor (effective capacity: 2 L), except the gas-solid separator (GSS), were 0.1 m, 0.05 m, and 0.4 m, respectively. The volume of a wastewater tank was 20 L, and the hydraulic retention time (HRT) was maintained at 24 hours. Neutrophilous granular sludge obtained from the treated brewery wastewater was used as the seed sludge of the UASB reactor. Before seeding the granular sludge, 5 mM terephthalic acid (TA), benzoic acid (BZ), p-toluic acid (PT), and AC were added to 1 L glass medium to enrich the aromatic compound degraded microorganism group in the seed sludge at 37° C. for one year under anaerobic conditions. The enriched sludges were seeded at an ignition loss (TVS) of 34 g, TVS of 3 g, and TVS of 14 g on Days 1, 11, and 45, respectively.

### Analytical Method

Wastewater samples were collected from the wastewater tank (influent) and effluent of the UASB reactor for routine analyses. The pH and biogas generation volume were measured on site. The pH was measured using a pH meter (HM-30P manufactured by DKK-TOA). The biogas generation volume was detected using a wet gas meter (W-NK-0.5B manufactured by Shinagawa Co., Ltd.). Total solids (TS) and TVS were measured by heating 20 milliliters of influent and effluent in an oven at 105° C. for 24 hours and 60° C. for 30 minutes, respectively. COD was analyzed with a spectrophotometer (HACH, DR1900, USA) using the potassium dichromate method. The biogas components of CH₄, CO₂, N₂, and H₂ were measured using gas chromatography (GC-8A manufactured by SHIMADZU CORPORATION) with a thermal conductivity detector fitted with a SHINCARBON-ST 50/80 stainless steel Column 4.0 m×3.0 mm (ID). The temperature of the column was 130° C., and the temperature of the injection port/detector was 150° C. The concentrations of the aromatic compounds (TA, IA, OA, PT, and BZ) of influent and effluent were measured by an ACQUITY UPLC H-Class system equipped with an ACQUITY UPLC BEH C18 (1.7 μm, 2.1×100 mm) column using a PDA detector (270 nm) (manufactured by Nihon Waters). All the aromatic compounds were detected at a flow rate of 0.4 mL/min, a column temperature of 35° C., solvent A 20% methanol, solvent B 100% methanol, solvent C 1.0% formic acid, gradient A:B:C=80:10:10 (0 to 5.0 min), A:B:C=30:60:10 (5.0 to 8.0 min), and A:B:C=80:10:10 (8.0 to 10 min). The volatile fatty acids (VFAs) of influent and effluent were analyzed by a Prominence HPLC system (post-column pH-buffered electrical conductivity detection system manufactured by SHIMADZU CORPORATION) attached to a Shim-pack Fast-OA column (100 mm L.×7.8 mm I.D., 5 μm, x2 in series) using a conductive detector. Prepared solvents A (5 mM p-toluenesulfonic acid) and B (5 mM p-toluenesulfonic acid 20 mM Bis-Tris, 0.1 mM EDTA) were purchased from SHIMADZU CORPORATION. The flow rates of pumps A and B were set at 0.8 mL/min, and a column temperature was set at 40° C.

### Continuous Treatment of PTA/DMT Manufacturing Wastewater

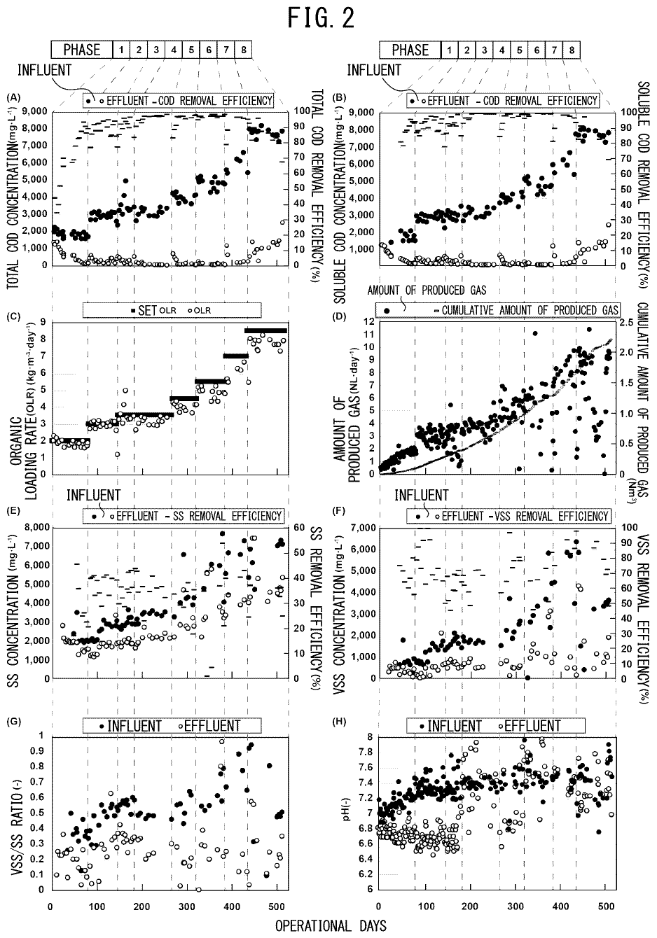

During the entire operational period, the pH values in the influent and effluent were 7.0±0.4 and 7.3±0.2, respectively (FIG. 2(H)). In the start-up period (phase 1), a total COD removal efficiency during Days 7 to 19 was 43±7.4%, while during Days 71 to 81, a total COD removal efficiency was 90±2.8% and a soluble COD removal efficiency was 94.3±1.0%, indicating that the seed sludges were acclimated to PTA/DMT wastewater degradation (FIGS. 2(A) and 2(B)). In phase 1, the total COD removal efficiency was 71±19%, and the soluble COD removal efficiency was 89±6.1% (Table 3). During phases 1 to 8, the organic loading rate (OLR) was increased from 1.8±0.2 kg·m⁻³ day⁻¹ to 7.6±0.7 kg·m⁻³·day⁻¹ and increased with substrate concentration (Tables 1 to 3). DMT and FM contained in the DMT manufacturing wastewater were added to the influent wastewater in the phases 3 and 4, respectively, and the concentrations were fixed according to known discharged levels after the phase 4 (NPLs 9 and 7) (Tables 1 and 2). The total COD removal efficiency and the soluble COD removal efficiency during phases 3 to 7 were 90% or more, indicating that the UASB reactor enables an OLR of 6.0±0.5 kg·m⁻³·day⁻¹. The produced biogas was composed of 72.8±3.5% CH₄, 24.2±3.4% CO₂, and 3.1±1.2% N₂ following the starting-up period (phases 2 to 8).

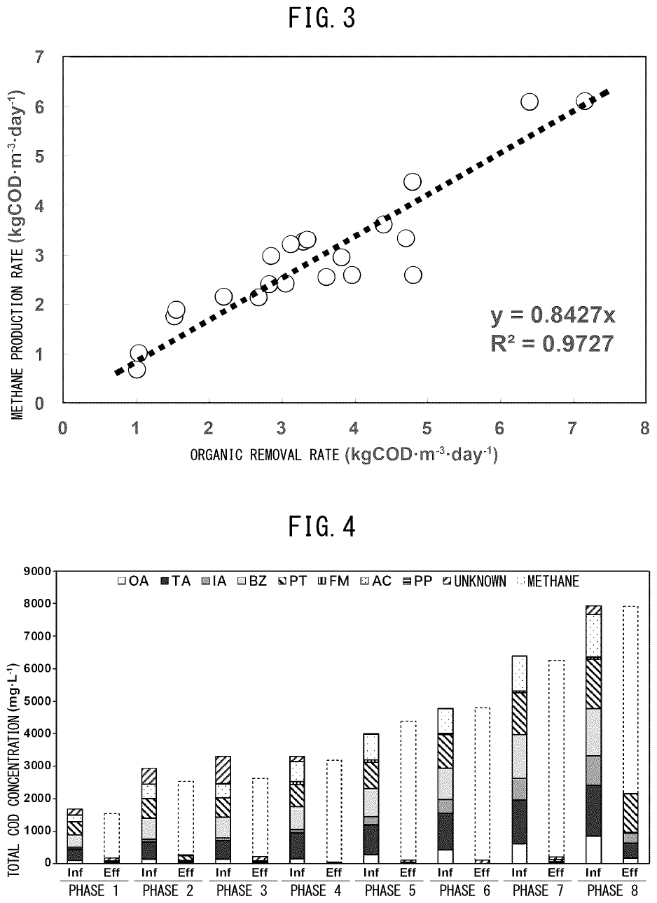

Based on the linear relationship (R²=0.973) between methane production rates and COD removal rates, the methane conversion efficiency was 84% in the UASB reactor (FIG. 3), which is comparable with previous study in the two-stage UASB system treating synthetic PTA wastewater containing AC, BZ, and TA as a methane conversion rate of more than 80% calculated from methane production rates and OLR (OLR of 25 kg·m⁻³ day⁻¹ and COD removal efficiency of 90%) (NPL 5). During the entire operational day, no H₂ gas was detected, indicating that VFA accumulation in the UASB reactor (propionic acid and the like), frequently occurring with high-OLR operation, did not occur in this study (NPL 13). At an OLR of 7.6±0.7 kg·m⁻³ day “1, the total COD and soluble COD removal efficiencies significantly dropped to 87% or less (Table 3). Aromatic compounds and organic acids in the influent wastewater were mostly removed by the UASB reactor during phases 2 to 7 (removal efficiency with respect to COD concentration: 76 to 100%), but high concentrations of PT (1,180 mgCOD/L, removal efficiency of 22%), TA (467 mgCOD/L, 70%), IA (315 mgCOD/L, 65%), and OA (160 mgCOD/L, 81%) remained in the wastewater at phase 8.

High concentrations of BZ and AC inhibit the degradation of TA and PT (NPLs 14 and 15). Moreover, PT is one of the compounds most resistant to biodegradation among the organic compounds in PTA wastewater (NPL 15). Furthermore, the biodegradability of phthalic acids in the natural environment is OA>TA>IA (NPL 16). In phase 8, the order of the removal efficiencies of aromatic compounds was BZ>OA>TA>IA>PT (FIG. 4), consistent with the previous studies described above. Based on these removal efficiencies, PT degradation might be competed with BZ degradation in the UASB reactor due to the lowest biodegradability in phase 8 with the highest BZ concentration (FIG. 4, Table 3, and Table 1). Accordingly, this study indicates that treatment of PT and the inhibitor thereof is the most critical step for successful treatment of PTA/DMT manufacturing wastewater under methanogenic conditions.

TABLE 3
Summary of Organic Loading Rate and Chemical Oxygen Demand Concentration
in Upflow Anaerobic Sludge Blanket (UASB) Reactor.

| | | OLR¹ | Total COD² (mg/L) | | Total COD | Soluble COD (mg/L) | | Soluble COD |
|---|---|---|---|---|---|---|---|---|
| phase | Days | kg (m³ · day) | Inf. | Eff. | rem.³ (%) | Inf.⁴ | Eff.⁵ | rem. (%) |
| 1 | 1-81 | 1.8 ± 0.2 | 1841 ± 197 | 617 ± 413 | 70.9 ± 19.2 | 1646 ± 252 | 498 ± 399 | 89.2 ± 6.1 |
| 2 | 82-146 | 2.7 ± 0.6 | 2948 ± 188 | 327 ± 135 | 89.0 ± 5.0 | 2827 ± 196 | 238 ± 117 | 91.4 ± 4.3 |
| 3 | 147-183 | 3.3 ± 1.0 | 3459 ± 726 | 305 ± 137 | 90.4 ± 6.1 | 2845 ± 305 | 234 ± 139 | 91.3 ± 6.2 |
| 4 | 184-268 | 3.1 ± 0.3 | 3134 ± 254 | 147 ± 141 | 95.3 ± 4.2 | 3089 ± 284 | 93 ± 141 | 97.1 ± 4.1 |
| 5 | 269-323 | 4.0 ± 0.2 | 3991 ± 230 | 219 ± 208 | 94.6 ± 4.6 | 3936 ± 267 | 161 ± 188 | 95.9 ± 4.9 |
| 6 | 324-386 | 4.8 ± 0.3 | 4829 ± 325 | 131 ± 50 | 97.3 ± 1.0 | 4716 ± 366 | 53.8 ± 53.5 | 98.9 ± 1.0 |
| 7 | 387-438 | 6.0 ± 0.5 | 5807 ± 669 | 421 ± 421 | 92.6 ± 7.6 | 5798 ± 665 | 333 ± 421 | 94.3 ± 7.2 |
| 8 | 439-518 | 7.6 ± 0.7 | 7616 ± 674 | 1087 ± 572 | 85.9 ± 7.2 | 7545 ± 674 | 1005 ± 561 | 86.9 ± 7.2 |

¹Organic loading rate
²Chemical oxygen demand
³Removal efficiency
⁴Influent
⁵Effluent

In this study, the organic removal rates in the UASB reactor were 5.6 kg·m⁻³ day⁻¹ in phase 7 and 6.6 kg·m 3 day⁻¹ in phase 8 (Table 3). The previously reported organic removal rates of a single anaerobic reactor treating PTA wastewater (except for hybrid process, process treating wastewater containing only TA, and no confirmation of TA degradation in the process) were 1.5 to 5.5 kg·m⁻³·day⁻¹ (NPLs 5, 12, and 17), and therefore, the UASB reactor in this study demonstrates the highest organic removal rate. The reason for this is considered to be the influence of MT and FM derived from DMT manufacturing wastewater. The increase of MT concentration and the addition of FM may activate MT-utilizing and/or hydrogenotrophic methanogenic archaea, respectively, in the UASB granular sludge. In order to reduce the TA/PT degradation inhibition by BZ and AC at high concentrations, two-stage treatment processes have been studied for treatment of PTA manufacturing wastewater.

Until now, the organic removal rates of two-stage UASB reactors and upflow anaerobic fluidized bed blanket-sequencing batch reactor (UAFB-SBR) systems have been reported to be 22.5 kg·m⁻³ day⁻¹ and 9 to 10 kg·m⁻³ day “1, respectively (NPLs 5 and 18). To enhance organic removal rates of more than 6.6 kg·m⁻³ day⁻¹ at phase 8, an additional treatment step for inhibitor removal is required. In addition, the PTA/DMT manufacturing wastewater in this study did not contain low concentrations of aromatic compounds (trimellitic acid, 4-carboxybenzaldehyde, and the like) (NPL 19) or formaldehyde derived from DMT manufacturing wastewater (NPL 9). Accordingly, it is necessary to confirm the biodegradability of these materials through reactor- and batch culture-based studies such as continuous treatment and methane activity studies. Overall, this study indicates that cotreatment of PTA manufacturing wastewater and DMT manufacturing wastewater is feasible and may remove an organic compound at a higher OLR than that in the case of PTA manufacturing wastewater alone. This is the first report on composite treatment of PTA manufacturing wastewater and DMT manufacturing wastewater using the UASB reactor.

1. A method of treating organic wastewater containing an aromatic compound, the method comprising:
(a) a step of performing addition of formic acid and methanol to the organic wastewater to make mixed wastewater; and
(b) a step of subjecting the mixed wastewater to anaerobic biological wastewater treatment to degrade an organic substance in the mixed wastewater.

2. The method according to claim 1, wherein the organic wastewater is purified terephthalic acid (PTA) manufacturing wastewater.

3. The method according to claim 1, wherein the addition of the formic acid and the methanol in the step (a) is performed by addition of dimethyl terephthalate (DMT) manufacturing wastewater.

4. The method according to claim 1, wherein the aromatic compound contained in the organic wastewater is one or two or more compounds selected from a group consisting of orthophthalic acid, benzoic acid, terephthalic acid, isophthalic acid, p-toluic acid, and salts and esters thereof.

5. A method of treating purified terephthalic acid (PTA) manufacturing wastewater, the method comprising:
(a) a step of adding dimethyl terephthalate (DMT) manufacturing wastewater to the PTA manufacturing wastewater to make mixed wastewater; and
(b) a step of subjecting the mixed wastewater to anaerobic biological wastewater treatment to degrade an organic substance in the mixed wastewater,
wherein the DMT manufacturing wastewater contains formic acid and methanol.

6. The method according to claim 5, wherein an aromatic compound contained in the PTA manufacturing wastewater is one or two or more compounds selected from a group consisting of orthophthalic acid, benzoic acid, terephthalic acid, isophthalic acid, p-toluic acid, and salts and esters thereof.

7. The method according to claim 1, wherein a concentration of the formic acid in the mixed wastewater prepared in the step (a) is 1 to 3% with respect to a total COD concentration.

8. The method according to claim 1, wherein a concentration of the methanol in the mixed wastewater prepared in the step (a) is 7 to 13% with respect to a total COD concentration.

9. The method according to claim 1, wherein the method further comprises (c) a step of separating, from the mixed wastewater, methane generated by degrading the organic substance in the step (b).

10. The method according to claim 1, wherein the anaerobic biological wastewater treatment in the step (b) is performed using a reactor filled with a methanogenic sludge.

11. The method according to claim 10, wherein wastewater is treated in upflow in the reactor filled with the methanogenic sludge.

12. The method according to claim 10, wherein the methanogenic sludge comprises methanogenic archaea and anaerobic symbiotic bacteria.

13. The method according to claim 12, wherein the methanogenic archaea comprise one or two or more kinds of archaea selected from a group consisting of archaea belonging to genera Methanobacterium, Methanolinea, Methanoregula, Methanothrix, Methanomethylovorans, and Methanomassiliicoccus.

14. The method according to claim 12, wherein the anaerobic symbiotic bacteria comprise one or two or more kinds of bacteria selected from a group consisting of bacteria belonging to genera Syntrophus, Syntrophorhabdus, and Pelotomaculum.
